(19) United States
(12) Patent Application Publication
(10) Pub. No.: US 2020/0087261 A1
(43) Pub. Date: Mar. 19, 2020

(54) METHOD FOR PREPARING DEUTERATED IMIDAZOLE DIKETONE COMPOUND

(71) Applicant: HINOVA PHARMACEUTICALS INC., Chengdu, Sichuan (CN)

(72) Inventors: Yuanwei CHEN, Chengdu, Sichuan (CN); Wu DU, Chengdu, Sichuan (CN); Tongtao KUANG, Chengdu, Sichuan (CN); Xi GENG, Chengdu, Sichuan (CN)

(21) Appl. No.: 16/342,912

(22) PCT Filed: Dec. 20, 2016
(86) PCT No.: PCT/CN2016/110978

§ 371 Date: Apr. 17, 2019

(30) Foreign Application Priority Data
Oct. 17, 2016 (CN) 201610901502.0

(51) Int. Cl. C07D 233/86; C07B 59/00

(52) U.S. Cl. CPC C07D 233/86; C07B 2200/05; C07B 59/002

(57) ABSTRACT

A method for preparation of deuterated imidazole diketone compounds includes the following steps: (1) using compound of formula (I) and compounds of formula (II) as starting material, compounds of formula (III) can be obtained by a substitution reaction; (2) Compounds of formula (IV) can be prepared by esterification of carboxyl in compounds of formula (III); (3) Cyclization of compounds of formula (IV) and compound of formula (V) provides compounds of formula (VI); (4) Compounds of formula (VI) are deesterificated and react to produce compounds of formula (VII); (5) using compounds of formula (VII) and compounds of formula (VIII) as starting material, the deuterated imidazole diketone compounds of formula (IX) are obtained by the condensation reaction of amide.

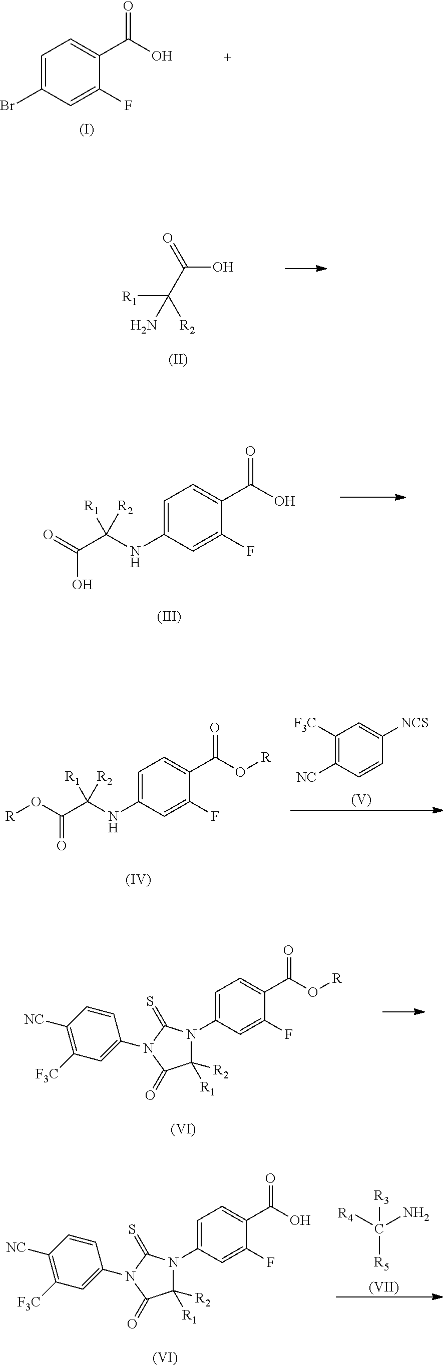

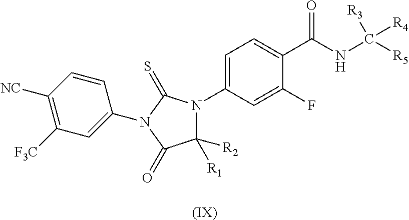

### TECHNICAL FIELD

The present invention relates to the field of medicinal synthesis, and particularly to a method for the preparation of deuterated imidazole diketone compounds.

### BACKGROUND ART

Prostatic cancer (PCa) is a common malignant tumor in male genital system, and the occurrence of this disease increases as the age, whose incidence rate has an obvious regional disparity, with Europe and America regions being higher. PCa is the second cancer causing male death, only next to lung cancer. In the past, in tumor spectrum of our country, PCa belongs to the minor disease and is not given enough attention. As the social development and progress of our country, the graying of society, the urbanization of human population, the westernization of dietary pattern, and the advancement of detection technology, the incidence rate of prostatic cancer in our country presents an obvious uptrend. The survey finished by the Second Affiliated Hospital of Tianjin Medical University and Tianjin Prostate Cancer Diagnosis and Treatment Cooperative Group in 2011 showed that the incidence rate of prostate cancer in Tianjin city was rapidly increasing, and the incidence rate of prostate cancer increased four times during the past 20 years. The patients with prostate cancer have already occupied 13.4% of urology tumor inpatients, and prostate cancer becomes a common tumor from the past rare cancer. In whole country, the incidence rate of prostate cancer presents the same trend.

Androgen is a ligand-dependent reverse transcription regulatory protein, with 1.1×10⁵ Dalton molecular weight. Androgen plays an very important role in the cause of prostate cancer and during its progression, as well as diseases related with male hormones such as acne, male lipsotrichia and so on.

The common treatment method for prostate cancer is surgery or androgen antagonists such as bicalutamide. However, after treatment for 2-4 years, patients can develop drug resistance, meanwhile bicalutamide further has an adverse effect of irritating cancer proliferation, and thus patients must stop using it. At present, compounds with the same bind target points as bicalutamide have already been developed, together with other drugs marketed for the treatment of metastatic prostate cancer, such as the patent CN201280052853.9.

Wherein, the following compound has better pharmacy character:

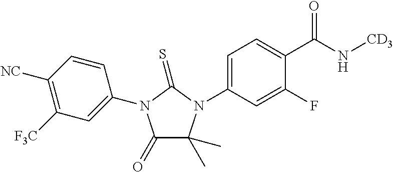 [SMILES: [2H]C([2H])([2H])NC(=O)c1ccc(N2C(=S)N(c3ccc(C#N)c(C(F)(F)F)c3)C(=O)C2(C)C)cc1F]

However, in the synthetic route disclosed in patent CN201280052853.9, isothiocyanate and isobutyronitrile were used for coupling. The main shortcoming in this method includes that only 11% yield of target product is obtained in the final step. That results in only 3.5% total yield of this route, beginning with the commercial available starting material. Moreover, the purification of each intermediate need use time-consuming and laborious column chromatography, and the total produce time is longer, that are not good for industrial production.

Thus, in order to improve the production efficiency and reduce the production cost, it is necessary to improve the synthetic method.

### Content of the Invention

In order to solve above problems, the present invention provides a method for the preparation of deuterated imidazole diketone compounds, and it includes the following steps:

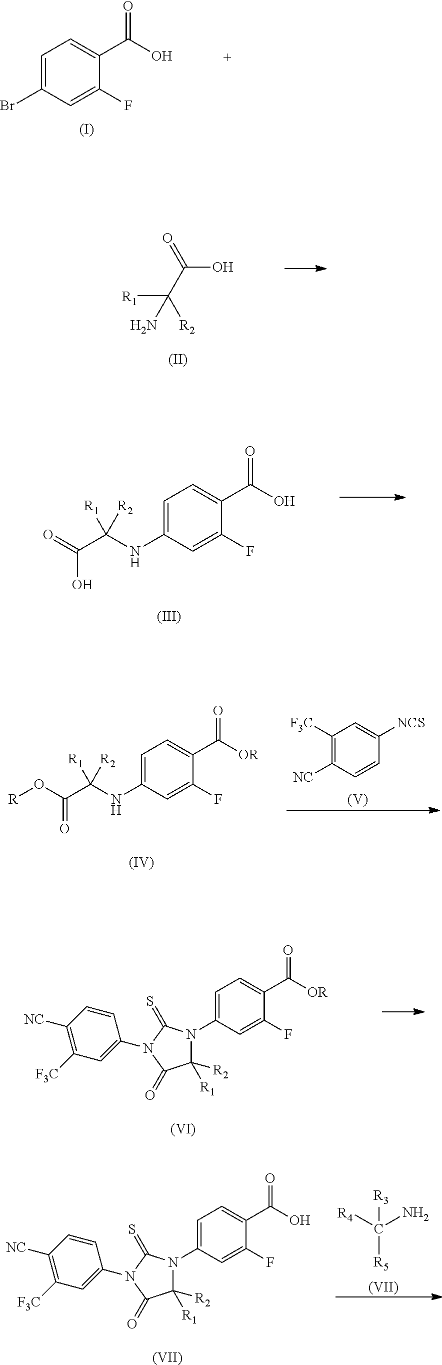

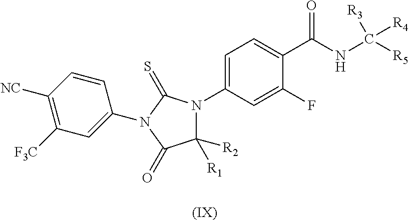

wherein R₁ and R₂ are independently selected from C₁-C₄ alkyls, or R₁ and R₂ link and form a ring together; R₃, R₄, and R₅ are selected from hydrogen or deuterium, in which at least one of them is selected from deuterium;
(1) using compound of formula (I) and compounds of formula (II) as starting material, compounds of formula (III) can be obtained by a substitution reaction;
(2) Compounds of formula (IV) can be prepared by esterification of carboxyl in compounds of formula (III), R is selected from C₁-C₆ alkyls;
(3) Cyclization of compounds of formula (IV) and compound of formula (V) provides compounds of formula (VI);
(4) Compounds of formula (VI) are deesterificated and react to produce compounds of formula (VII);
(5) using compounds of formula (VII) and compounds of formula (VIII) as starting material, the deuterated imidazole diketone compounds of formula (IX) are obtained by the condensation reaction of amide.

Further, R₁ and R₂ are both methyl.

Further, R₃, R₄ and R₅ are all deuterium.

Further, R is methyl. Further, in the cyclization of step (3), solvents are the mixture of dimethyl sulfoxide and isopropyl acetate.

Further, the volume ratio of dimethyl sulfoxide and isopropyl acetate is 1:2.

Further, in step (4), said reaction is carried out in the presence of base, and the base is selected from the alkali metal hydroxides.

Further, said alkali hydroxides are selected from LiOH, KOH, and NaOH, and preferably LiOH.

Further, in step (5), said amide condensation reaction is carried out in the presence of condensing agent, and the condensing agent is selected from isopropyl chlorocarbonate, N,N′-carbonyldiimidazole, or HATU.

Further, in step (1), said substitution reaction is carried out in alkaline environment in the presence of Cu, CuI, and N,N-dimethylglycine.

Further, in step (2), said methyl esterification reagent for carboxyl is methyl iodide.

Said C₁-C₄ alkyls denote C₁, C₂, C₃, C₄ alkyls, i.e. straight chain or branch chain alkyls having 1-4 carbon atoms, such as methyl, ethyl, propyl, isopropyl, butyl, isobutyl, tert-butyl, sec-butyl, etc.

Compared with the methods in the prior art, the present invention has the following obvious advantages:

(1) The method of the present invention can realize the total yield of 40%, greatly better than 3.5% total yield of the current method.
(2) For the method of the present invention, the procedures are simple, not needing column chromatography, and only simple precipitation or crystallization means can perform the purification of products.
(3) The method of the present invention avoids the use of extremely toxic reagent acetone cyanohydrin, and is more green and safe.

The inventors explore another synthetic route in the research and development process. By introducing deuterium source in the earlier stage, trideuteromethylbenzamide is obtained, then 2-(3-fluoro-4-(trideuteromethylformamyl)phenylamine)-2-methylpropionic acid is obtained by the condensation of trideuteromethylbenzamide and 2-methylalanine in the presence of copper catalyst. Propionic acid is methylated and finally cyclized with 4-isothiocyanato-2-(trifluoromethyl)benzonitrile to provide the target compound.

The present invention further provides a method for the preparation of deuterated imidazole diketone compounds, and it includes the following steps:

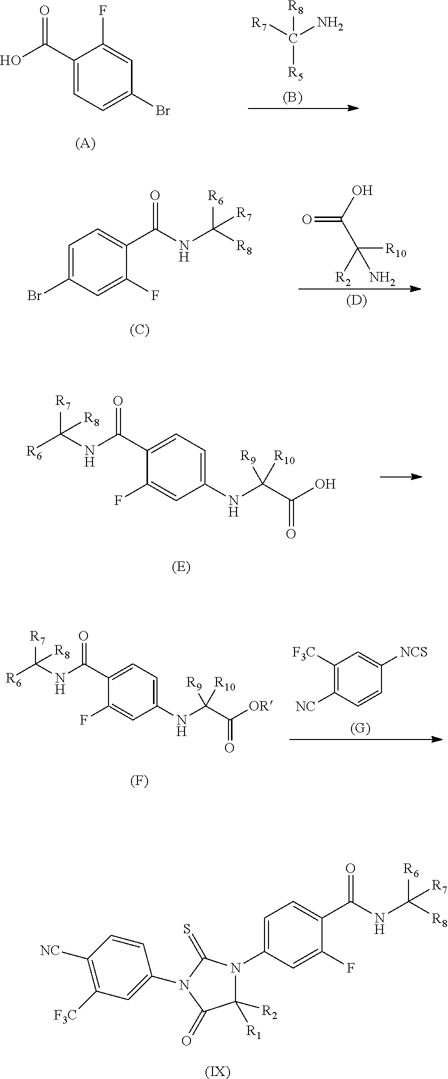

Wherein, R₆, R₇, and R₅ are selected from hydrogen or deuterium, and at least one of them is selected from deuterium; R₉ and R₁₀ are independently selected from C₁-C₄ alkyls, or R₉ and R₁₀ link and form a ring together;

(1) Using compound of formula (A) and compound of formula (B) as starting material, compound of formula (C) is obtained by amide condensation reaction;
(2) Using compound of formula (C) and compound of formula (D) as starting material, compound of formula (E) is obtained by substitution reaction;
(3) Compound of formula (F) is prepared by esterification of carboxyl in compound of formula (E), and R′ is selected from C₁-C₆ alkyls;
(4) The deuterated imidazole diketone compounds of formula (IX) is obtained by cyclization of compound of formula (F) and compound of formula (G).

Further, R₉ and R₁₀ are both methyl.

Further, R₆, R₇ and R₈ are all deuterium.

Further, R′ is methyl.

Compared with above-mentioned route, this route has one less step, but considering deuterated reagents being more expensive, introducing deuterium source at earlier stage and the loss of yield by several synthetic steps, the total cost is finally higher than that of introducing deuterium source in the final step.

In the present invention, the abbreviations are listed in the following Table:

| | |
|---|---|
| DMSO | dimethyl sulfoxide |
| IPAc | isopropyl acetate |
| DMF | N,N-dimethylformamide |
| EA | ethyl acetate |
| THF | tetrahydrofuran |
| DMAA | N,N-dimethylglycine |
| HATU | 2-(7-oxybenzotriazole)-N,N,N′,N′-tetramethylurea |
| | hexafluorophosphate |
| CDI | N,N′-carbonyldiimidazole |

Obviously, based on above content of the present invention, according to the common technical knowledge and the conventional means in the field, without department from above basic technical spirits, other various modifications, alternations or changes can further be made.

By following specific examples of said embodiments, above content of the present invention is further illustrated. But it should not be construed that the scope of above subject of the present invention is limited to following examples. The techniques realized based on above content of the present invention are all within the scope of the present invention.

### EXAMPLES

### Example 1 Preparation of 4-(1-carboxyl-1-methyl-ethylamino)-2-fluoro-benzoic Acid

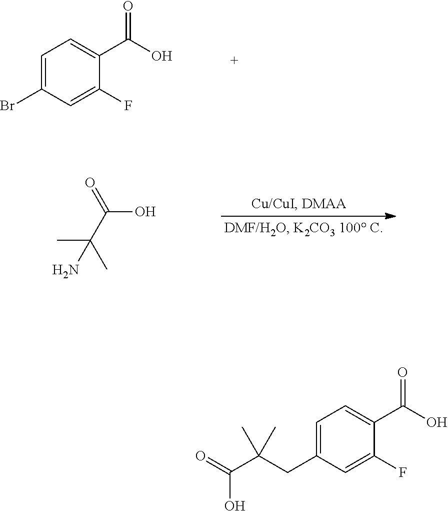

To the reaction kettle, was introduced nitrogen gas, to which were added N,N-dimethylformamide (20 L), water (2 L), 4-bromo-2-fluorobenzoic acid (2.0 kg), 2-methylalanine (2.82 kg), N,N-dimethylglycine (474 g), potassium carbonate (6.31 kg), copper powder (116 g), and CuI (348 g). Under the protection of nitrogen, the mixture was stirred at 110° C. for 16 hours. The reaction solution was cooled to the room temperature, and then ice water (30 L) was added. The pH value of solution was adjusted to 4-5 by drop adding 6N ice hydrochloric acid, and extracted with ethyl acetate twice (20 L*2). The organic phase was combined, and washed with water (5 L) once. The organic phase was concentrated to dry, to which was added dichloromethane (15 L) for pulping, then filtered, dried in vacuum at 40° C., to provide the target compound 1.797 kg, with a yield of 81.7%.

### Example 2 Preparation of 2-fluoro-4-[(1-methoxyl-2-methyl-1-oxo-2-propyl)amino]benzoic Acid Methyl Ester

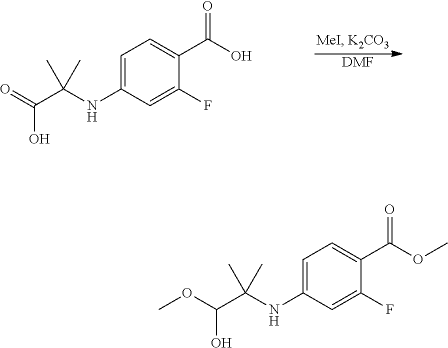

To the reaction kettle, were added N,N-dimethylformamide (7 L), 4-(1-carboxyl-1-methyl-ethylamino)-2-fluoro-benzoic acid (750 g), and potassium carbonate (6.31 kg). Then, methyl iodide (945 g) was added, and the mixture was stirred overnight. To the reaction solution, was drop added water (20 L), and stirred for crystallization, filtered, dried in vacuum at 50° C. for 16 hours, to provide the target compound 787 g, with a yield of 93.9%.

¹HNMR (DMSO, 400 MHz): 1.49 (6H, s), 3.64 (3H, s), 3.75 (3H, s), 6.16 (1H, dd, J=2.1, 14.5 Hz), 6.31 (1H, dd, J=2.1, 8.8 Hz), 7.16 (1H, s), 7.63 (1H, t, J=8.8 Hz).

### Example 3 Preparation of 4-{3-[4-cyano-3-(trifluoromethyl)phenyl]-5,5-dimethyl-4-oxo-2-thio-1-imidazolidinyl}-2-fluorobenzoic Acid Methyl Ester

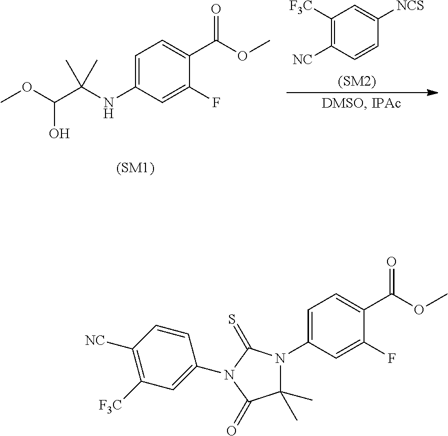

To the reaction kettle, were added 2-fluoro-4-[(1-methoxyl-2-methyl-1-oxo-2-propyl)amino]benzoic acid methyl ester (1.70 kg), 4-isothiocyanato-2-(trifluoromethyl)benzonitrile (2.88 kg), dimethyl sulfoxide (1.7 L), and iso-propyl acetate (3.4 L). Under the protection of nitrogen, the mixture was stirred at 83° C. for 40 hours. The reaction solution was concentrated under reduced pressure till no solvent was evaporated, then methanol (8.5 L) was drop added, and stirred at 0-5° C. for crystallization, filtered. The filter cake was washed with methanol and dried in vacuum at 50° C. to provide the target compound 2.3 kg, with a yield of 78.2%.

¹HNMR (DMSO, 400 MHz): 1.57 (6H, s), 3.91 (3H, s), 7.43 (1H, dd, J=1.8, 8.3 Hz), 7.53 (1H, dd, J=1.8, 11.2 Hz), 8.08-8.12 (2H, m), 8.30 (1H, d, J=1.5 Hz), 8.42 (1H, d, J=8.2 Hz).

According to above method, the inventors investigated the reaction solvents, and results are shown in Table 1:

TABLE 1
Investigation of reaction solvents

| | | Amount(g) | | | | | | | |
|---|---|---|---|---|---|---|---|---|---|
| | | Experiment 1 | | Experiment 2 | | Experiment 3 | | Experiment 4 | |
| Name | | (DMSO) | | (DMSO/IPAc) | | (DMF) | | (DMSO/EA) | |
| Experimental | SM1 | 2 | g | 2 | g | 2 | g | 2 | g |
| part | SM2 | 3.4 | g | 3.4 | g | 3.4 | g | 3.4 | g |

| | | | | | | |
|---|---|---|---|---|---|---|
| DMSO | 3 | ml | 2 | ml | — | — |

| | | | | | |
|---|---|---|---|---|---|
| IPAc | — | 4 | ml | — | — |

| | | | | | | |
|---|---|---|---|---|---|---|
| DMF | — | — | 3 | ml | 2 | ml |

| | | | | | |
|---|---|---|---|---|---|
| EA | — | — | — | 4 | ml |

| | | | | | |
|---|---|---|---|---|---|
| Conclusion | Yield (%) | 57.8% | 82.3% | 29% | 49.1% |
| Evaluation | mass | 98% | 92% | 98% | 98% |
| | (LCMS) | | | | |
| | (HPLC) | | | | |

It can be shown that when the solvent is the mixture of dimethyl sulfoxide and iso-propyl acetate, the yield is higher.

### Example 4 Preparation of 4-{3-[4-cyano-3-(trifluoromethyl)phenyl]-5,5-dimethyl-4-oxo-2-thio-1-imidazolidinyl}-2-fluorobenzoic Acid

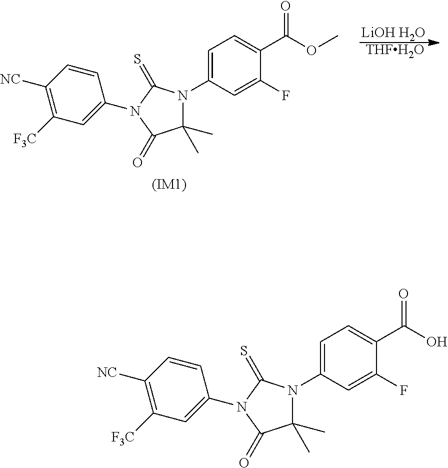

To the reaction kettle, was introduced nitrogen gas, to which were added 4-{3-[4-cyano-3-(trifluoromethyl)phenyl]-5,5-dimethyl-4-oxo-2-thio-1-imidazolidinyl}-2-fluorobenzoic acid methyl ester (1.70 kg) and tetrahydrofuran (3.4 L), and the mixture was stirred till the solution was clear. Lithium hydroxide aqueous solution (lithium hydroxide monohydrate 0.46 kg+water 3.4 L) was drop added. At the temperature of 40° C., the mixture was incubated for 1 h. The mixture was cooled to the temperature, and water (3.4 L) was added, then the pH value was adjusted to 1-2 using brine. Ethyl acetate (13.6 L) was added for extraction. The organic phase was washed with water once, dried with anhydrous sodium sulfate, filtered, and concentrated under reduced pressure to about 3.4 L solvent remained. n-heptane (10.2 L) was added and stirred for crystallizing 1 h, then filtered and dried in vacuum to provide the target compound 1.31 kg, with a yield of 79.4%.

¹HNMR (DMSO, 400 MHz): 1.57 (6H, s), 7.39 (1H, dd, J=1.6, 8.3 Hz), 7.48 (1H, dd, J=1.6, 11.0 Hz), 8.06-8.12 (2H, m), 8.32 (1H, d, J=1.2 Hz), 8.42 (1H, d, J=8.2 Hz), 13.58 (1H, brd).

According to above method, the inventors investigated the bases, and results are shown in Table 2:

TABLE 2
Investigation of bases

| | | Amount (g) | | | | |
|---|---|---|---|---|---|---|
| | | Experiment 1 | Experiment 2 | Experiment 3 | Experiment 4 | Experiment 4 |
| Name | | (KOH) | (NaOH) | (LiOH•H₂O) | (K₂CO₃) | (Na₂CO₃) |
| Experimental | IM1 | 4.44 g | 2 g | 2 g | 2 g | 2 g |
| part | base | (3 eq) 1.88 g | (3 eq) 516 mg | (3 eq) 543 mg | (3 eq) 1.76 g | (3 eq) 1.37 g |
| | THF | 10 ml | 5 ml | 5 ml | 5 ml | 5 ml |
| | water | 5 ml | 7 ml | 7 ml | 7 ml | 7 ml |
| Conclusion | Yield (%) | 3.9 g, 89.4% | 1.988 g, 100% | 2.01 g, 87% | — | — |
| Evaluation | Mass | 90.7% | 93.62% | 97.46% | 0% | 0% |
| | (LCMS) | | | | | |
| | (HPLC) | | | | | |

It can be found that potassium carbonate and potassium carbonate were basically not reacted, and when the base is selected from the alkali metal hydroxides, the yield is higher. Amongst, the yield is higher with LiOH, and the purity is also higher, and LiOH is preferable.

### Example 5 Preparation of 4-{3-[4-cyano-3-(trifluoromethyl)phenyl]-5,5-dimethyl-4-oxo-2-thio-1-imidazolidinyl}-2-fluoro-N-trideuteromethylbenzamide

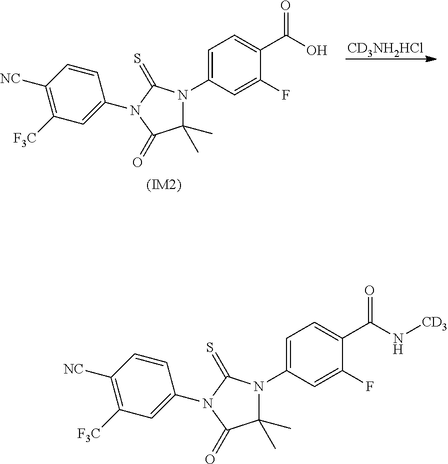

To the reaction kettle, were added 4-{3-[4-cyano-3-(trifluoromethyl)phenyl]-5,5-dimethyl-4-oxo-2-thio-1-imidazolidinyl}-2-fluorobenzoic acid (1.30 kg) and dichloromethane (13 L), and the mixture was stirred for dissolution. N,N′-carbonyldiimidazole (0.7 kg) was added in batches, and stirred for 2 h. Triethylamine (1.2 L) and deuterated methylamine hydrochlorate (302 g) were added, and stirred for 4 hours at room temperature. The reaction solution was successively washed with 1 N sodium hydroxide aqueous solution (13 L), 1 N hydrochloric acid (13 L), and water (6.5 L). The organic phase was dried with anhydrous sodium sulfate, concentrated under reduced pressure to just precipitate solids. Methyl tert-butyl ether (3.9 L) and n-heptane (3.9 L) were successively drop added, stirred, precipitated for 1 h, and filtered, to obtain crude products. The crude products were dissolved in absolute alcohol (13 L) under heating, and at the temperature of 0-5° C., the solution was stirred and crystallized for 2 h, then filtered. At the temperature of 50° C., the crystal was dried in vacuum for 8 hours, to provide the target compound as white solid (1.09 kg), with a yield of 80.7%. The purity was 99.8% by HPLC.

¹HNMR (DMSO, 400 MHz): 1.57 (6H, s), 7.36 (1H, dd, J=1.2, 8.2 Hz), 7.46 (1H, dd, J=1.2, 10.7 Hz), 7.82 (1H, t, J=8.2 Hz), 8.11 (1H, d, J=8.2 Hz), 8.32 (1H, s), 8.42 (1H, d, J=8.2 Hz), 8.46 (1H, s).

According to above method, the inventors investigated the condensing agents, and results are shown in Table 3:

TABLE 3
Investigation of condensing agents

| | Amount (g) | | | | | | | | | |
|---|---|---|---|---|---|---|---|---|---|---|
| | Experiment 1 | | | | | | | | | |
| | (isopropyl | | Experiment 2 | | Experiment 3 | | Experiment 4 | | Experiment 5 | |
| Name | chlorocarbonate) | | (HATU) | | (CDI) | | (EDCI + HOBT) | | (dichlorosulfoxide) | |
| condensing | 0.22 | g (1.6 mmol) | 0.3 | g | 0.13 | g | 0.15 g + 0.11 g | | 0.06 | g (0.49 mmol) |
| agents | | | | | | | | | | |
| IM2 | 0.5 | g (1.1 mmol) | 0.18 | g | 0.18 | g | 0.18 | g | 0.2 | g (0.4 mmol) |
| methylamine | 0.15 | g (2.2 mmol) | 0.1 | g | 0.1 | g | 0.1 | g | 0.06 | g |
| hydrochlorate | | | | | | | | | | |
| triethylamine | 0.45 | g (4.4 mmol) | 0.33 | mL | 0.33 | mL | 0.3 3 | mL | 0.16 | g |

| | | | | | | |
|---|---|---|---|---|---|---|
| THF | 5 ml + 5 ml | — | — | — | 5 | mL |

| | | | | | | | | |
|---|---|---|---|---|---|---|---|---|
| DCM | — | 3 | mL | 3 | mL | 3 | mL | — |

| | | | | | |
|---|---|---|---|---|---|
| conclusion | IM2 remained | IM2 remained | IM2 remained | IM2 remained | no reaction, and |
| | about 1% | about 6.6%, and | about 1.79% | about 34% | heating caused the |
| | (according to the | reaction solution | (according to the | (according to the | solution becoming |
| | spectra) | was more complex | spectra) | spectra) | complex |
| | | (according to the | | | (according to TLC) |
| | | spectra) | | | |

It can be shown that when isopropyl chlorocarbonate or CDI is used as the condensing agent, the yield is higher. Amongst, isopropyl chlorocarbonate need reduce the temperature to form the mixed anhydride, and thus the operation has a high standard for the equipment. Comprehensively considering, the condensing agent is preferably CDI.

### Example 6

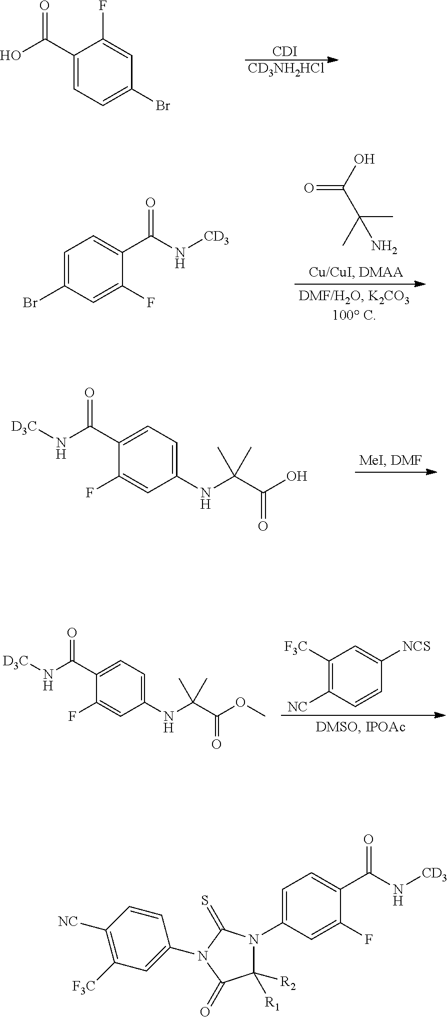

### (1) Preparation of 4-isothiocyanato-2-(trifluoromethyl)benzonitrile

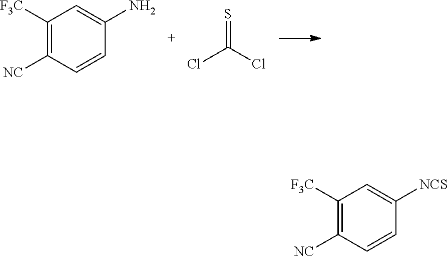

To the reaction kettle, was introduced nitrogen gas, to which were added 4-cyano-3-(trifluoromethyl)phenylamine (200 g), n-heptane (450 mL), and water (500 mL), followed by stirring to produce suspension. Thiophosgene (148 g) was drop added. The mixture was stirred at 40° C. for 16 hours, then stood for liquid separation. The water phase was extracted with n-heptane (500 mL) once, and the organic phase was combined. The solvent was removed by concentrating under reduced pressure, followed by reduced pressure distillation to provide the target compound (220 g), with a yield of 89.8%.

¹HNMR (DMSO, 400 MHz): 7.52 (1H, dd, J=1.7, 8.3), 7.60 (1H, d, J=1.7 Hz), 7.87 (1H, d, J=8.3 Hz).

### (2) Preparation of 4-bromo-2-fluoro-N-trideuteromethylbenzamide

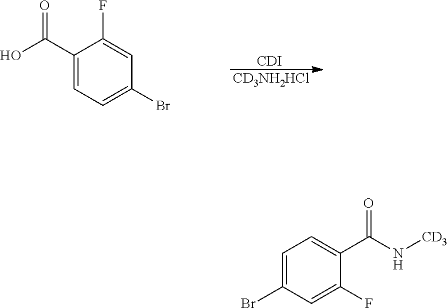 [SMILES: O=C(O)c1ccc(Br)cc1F ; "CD3NH2HCl" ; "CDI" ; [2H]C([2H])([2H])NC(=O)c1ccc(Br)cc1F]

To the reaction kettle, were added 4-bromo-2-fluorobenzoic acid (50 g) and dichloromethane (500 mL), then stirred. N,N-carbonyldiimidazole (73.9 g) was added in batches, and stirred for 2 h. Triethylamine (95.5 mL) and deuterated methylamine hydrochloride (30.8 g) were added and stirred for 4 hours. The reaction solution was sequentially washed with 1 N sodium hydroxide aqueous solution (500 mL), 1 N hydrochloric acid (500 mL), and water (250 mL). The organic phase was dried with anhydrous sodium sulfate, filtered, concentrated under reduced pressure to obtain the off-white solids, that was dried 8 hours to provide the target compound as off-white solids (44.0 g), with a yield of 82.2%.

### (3) Preparation of 2-(3-fluoro-4-(trideuteromethylformamyl)phenylamino)-2-methylpropionic Acid

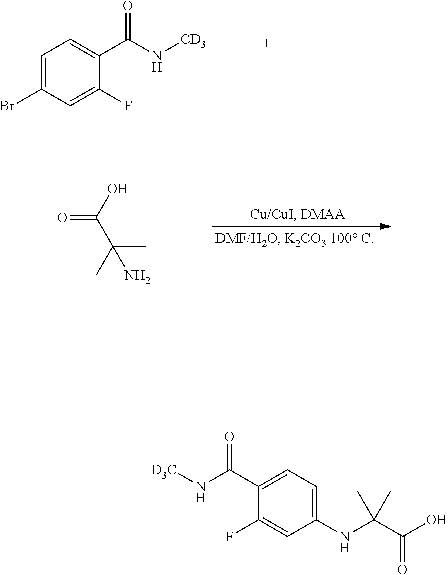

To the reaction kettle, was introduced nitrogen gas, to which were added N,N-dimethylformamide (680 mL), water (70 mL), 4-bromo-2-fluoro-N-trideuteromethylbenzamide (150 g), 2-methylalanine (199.8 g), N,N-dimethylglycine (33.3 g), potassium carbonate (446.1 g), copper powder (8.3 g), and CuI (24.6 g). Under the protection of nitrogen, the mixture was stirred at 110° C. for 16 hours. The reaction solution was cooled to the room temperature, and then water (1.8 L) was added. The impurities were extracted with ethyl acetate. The pH value of solution was adjusted to 3-4 using citric acid, crystallized for 1 hour at 5° C., filtered, and dried in vacuum to provide the target compound (100 g), with a yield of 60.9%.

### (4) 2-(3-fluoro-4-(trideuteromethylformamyl)phenylamino)-2-methylpropionic Acid Methyl Ester

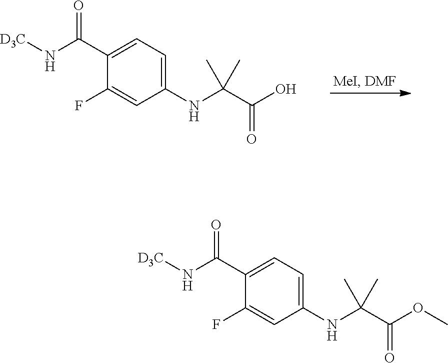 [SMILES: [2H]C([2H])([2H])NC(=O)c1ccc(NC(C)(C)C(=O)O)cc1F ; [2H]C([2H])([2H])NC(=O)c1ccc(NC(C)(C)C(=O)OC)cc1F]

To the reaction kettle, were added N,N-dimethylformamide (630 mL), 2-(3-fluoro-4-(trideuteromethylformamyl)phenylamino)-2-methylpropionic acid (90 g), water (2.2 mL), and potassium carbonate (58.7 g). Then, methyl iodide (26.5 mL) was added, and the mixture was stirred at 40° C. for 3 hours. To the reaction solution, was added acetic acid (6.4 mL), and stirred at 60° C. for 1 hour. Water (1.35 L) was drop added, cooled to the room temperature, stirred for crystallization, filtered, dried in vacuum, to provide the target compound 91 g, with a yield of 95.2%.

### (5) 4-{3-[4-cyano-3-(trifluoromethyl)phenyl]-5,5-dimethyl-4-oxo-2-thio-1-imidazolidinyl}-2-fluoro-N-trideuteromethylbenzamide

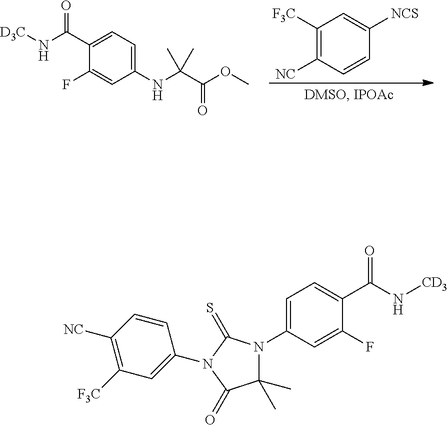

To the reaction kettle, was introduced nitrogen gas, to which were added 2-(3-fluoro-4-(trideuteromethylformamyl)phenylamino)-2-methylpropionic acid methyl ester (27.1 g), 4-isothiocyanato-2-(trifluoromethyl)benzonitrile (45.6 g), dimethyl sulfoxide (27.1 mL), and iso-propyl acetate (54.2 mL). Under the protection of nitrogen, the mixture was stirred at 83° C. for 24 hours. The reaction solution was concentrated under reduced pressure till no solvent was evaporated, then methanol (135.5 mL) was drop added, and stirred at 0-5° C. for 1 hour to crystallize, and filtered to obtain the crude product. The crude product was dissolved in absolute alcohol (250 mL) under heating and crystallized at 0-5° C. for 1 hour, filtered, and the filter cake was dried in vacuum at 50° C. to provide the target compound 34.5 g, with a yield of 73.9%.

In summary, compared with the methods in the prior art, the method according to the present invention is safer, consuming less solvents, minimizing the waste and the effect on the environment, shortening the production cycle, and improving the throughput and the total yield of the method, with a wide market outlook.

1. A method for the preparation of deuterated imidazole diketone compounds, characterized in that it includes the following steps: 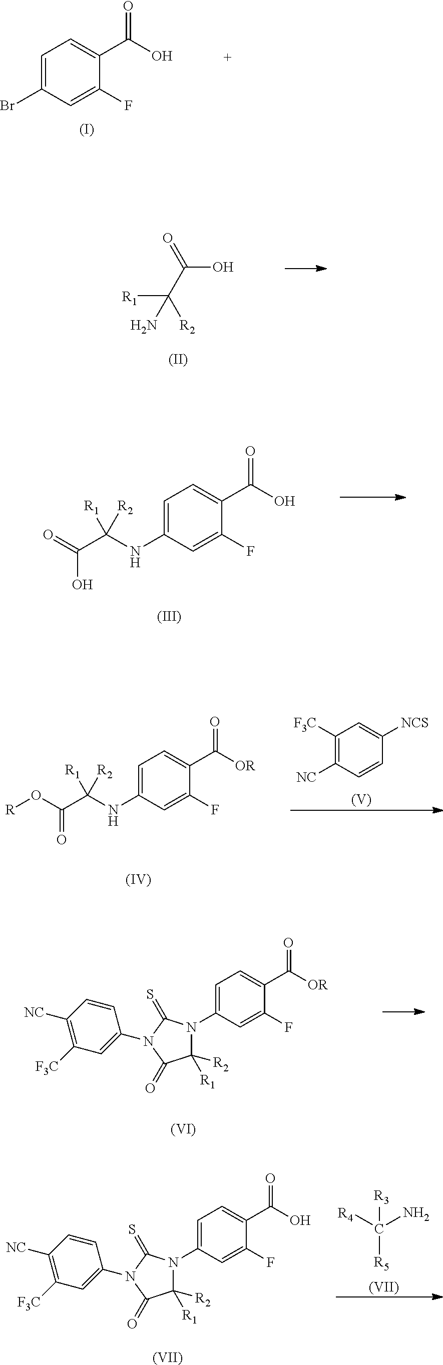 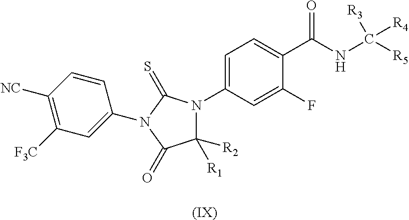
wherein R₁ and R₂ are independently selected from C₁-C₄ alkyls, or R₁ and R₂ link and form a ring together;
R₃, R₄, and R₅ are selected from hydrogen or deuterium, in which at least one of them is selected from deuterium; (1) using compound of formula (I) and compounds of formula (II) as starting material, compounds of formula (III) can be obtained by a substitution reaction;
(2) Compounds of formula (IV) can be prepared by esterification of carboxyl in compounds of formula (III), R is selected from C₁-C₆ alkyls;
(3) Cyclization of compounds of formula (IV) and compound of formula (V) provides compounds of formula (VI);
(4) Compounds of formula (VI) are deesterificated and react to produce compounds of formula (VII);
(5) using compounds of formula (VII) and compounds of formula (VIII) as starting material, the deuterated imidazole diketone compounds of formula (IX) are obtained by the condensation reaction of amide.

2. The method according to claim 1, characterized in that R₁ and R₂ are both methyl.

3. The method according to claim 1, characterized in that R₃, R₄ and R₅ are all deuterium.

4. The method according to claim 1, characterized in that R is methyl.

5. The method according to claim 1, characterized in that in step (1), the reaction temperature is 40-120° C.

6. The method according to claim 1, characterized in that in step (2), the reaction temperature is −10-60° C.

7. The method according to claim 1, characterized in that in step (3), the reaction temperature is 40-90° C.

8. The method according to claim 1, characterized in that in step (4), the reaction temperature is −10-70° C.

9. The method according to claim 1, characterized in that in step (5), the reaction temperature is −10-40° C.

10. The method according to claim 1, characterized in that in the cyclization of step (3), solvents are dimethyl sulfoxide or the mixture of dimethyl sulfoxide and isopropyl acetate, in which the volume ratio of dimethyl sulfoxide and isopropyl acetate is 50:1-1:10.

11. The method according to claim 10, characterized in that the volume ratio of dimethyl sulfoxide and isopropyl acetate is 1:2.

12. The method according to claim 1, characterized in that in step (4), said reaction is carried out in the presence of base, and the base is selected from the alkali metal hydroxides.

13. The method according to claim 12, characterized in that said alkali hydroxides are selected from LiOH, KOH, and NaOH, and preferably LiOH.

14. The method according to claim 1, characterized in that in step (5), said amide condensation reaction is carried out in the presence of condensing agent, and the condensing agent is selected from isopropyl chlorocarbonate, N,N′-carbonyldiimidazole, or HATU.

15. The method according to claim 1, characterized in that in step (1), said substitution reaction is carried out in alkaline environment, using Cu, CuI, and N,N-dimethylglycine as catalyst.

16. The method according to claim 1, characterized in that in step (2), said methyl esterification reagent for carboxyl is methyl iodide.

17. A method for the preparation of deuterium-substituted imidazole diketone compounds, characterized in that it includes the following steps: 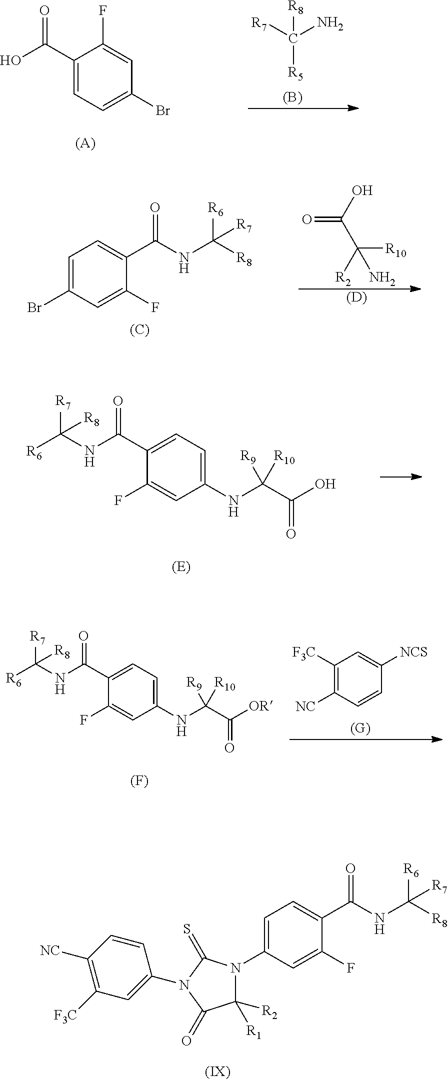
Wherein, R₆, R₇, and R₈ are selected from hydrogen or deuterium, and at least one of them is selected from deuterium; R₉ and R₁₀ are independently selected from C₁-C₄ alkyls, or R₉ and R₁₀ link and form a ring together;
(1) Using compound of formula (A) and compound of formula (B) as starting material, compound of formula (C) is obtained by amide condensation reaction;
(2) Using compound of formula (C) and compound of formula (D) as starting material, compound of formula (E) is obtained by substitution reaction;
(3) Compound of formula (F) is prepared by esterification of carboxyl in compound of formula (E), and R′ is selected from C₁-C₆ alkyls;
(4) The deuterated imidazole diketone compounds of formula (IX) is obtained by cyclization of compound of formula (F) and compound of formula (G).

18. The method according to claim 17, characterized in that R₉ and R₁₀ are both methyl.

19. The method according to claim 17, characterized in that R₆, R₇ and R₈ are all deuterium.

20. The method according to claim 17, characterized in that R′ is methyl.
